(19) United States
(12) Patent Application Publication
(10) Pub. No.: US 2017/0348015 A1
(43) Pub. Date: Dec. 7, 2017

(54) METHODS AND SYSTEMS FOR REMOVING TISSUE FROM BODY CAVITIES

(71) Applicant: PERCSYS SECURED CREDITORS, LLC, Monterey, CA (US)

(72) Inventor: Robert S. BEHL, Monterey, CA (US)

(21) Appl. No.: 15/660,423

(22) Filed: Jul. 26, 2017

Related U.S. Application Data
(63) Continuation of application No. 15/004,169, filed on Jan. 22, 2016, now abandoned.
Application No. 15/004,169 is a continuation-in-part of application No. 14/622,114, filed on Feb. 13, 2015, now abandoned.
Application No. 14/622,114 is a continuation of application No. 12/628,382, filed on Dec. 1, 2009, now Pat. No. 8,986,291.
(60) Provisional application No. 61/170,055, filed on Apr. 16, 2009.
(60) Provisional application No. 61/118,802, filed on Dec. 1, 2008.

(51) Int. Cl. A61B 17/221; A61B 1/00; A61B 1/018; A61B 17/22; A61B 18/26; A61B 1/307; A61B 90/00; A61B 17/00; A61B 18/24

(52) U.S. Cl. CPC A61B 17/221; A61B 18/26; A61B 1/00087; A61B 1/018; A61B 1/307; A61B 17/2202; A61B 2017/00296; A61B 2017/00287; A61B 18/24; A61B 1/00085; A61B 2017/22014; A61B 2017/22035; A61B 2017/22072; A61B 2090/3614; A61B 2217/007; A61B 2017/2215

(57) ABSTRACT

A stone or tissue capture device comprises a shaft with a deployable sweeping/containment structure at its distal end. The shaft is adapted to be removably placed over and connected to a conventional endoscope. The combination of the capture device and endoscope can be introduced into the various body lumens to capture, fragment/excise, and remove stones or tissue from the bladder and kidney, stomach, peritoneum, and from lumens such as the ureter, colon, hepatic ducts, airways, or blood vessels.

### CROSS-REFERENCE

This application is a continuation of U.S. patent application Ser. No. 15/004,169, filed Jan. 22, 2016, which is a continuation-in-part application of Ser. No. 14/622,114, filed Feb. 13, 2015, which is a continuation of Ser. No. 12/628,382, filed Dec. 1, 2009 and now issued as U.S. Pat. No. 8,986,291 on Mar. 24, 2015, which claims the benefit of Provisional Application No. 61/118,802, filed Dec. 1, 2008, and Provisional Application No. 61/170,055, filed on Apr. 16, 2009, the disclosures of which are incorporated herein by reference in their entirety.

### BACKGROUND OF THE INVENTION

### 1. Field of the Invention

The present invention relates generally to medical apparatus and methods. More particularly, the present invention relates to a method and apparatus for capturing, fragmenting, and removing urinary stones from the kidney or bladder as well as capturing, dissecting or incising, and removing from the interior of a kidney or bladder, or other body lumen.

Urinary tract endoscopy, a minimally invasive procedure for removing urinary stones, debulking growths and tumor, and collecting biopsy samples that are present in the bladder, ureter, or kidney, may be performed in several ways. A large viewing scope, referred to as a cystoscope, is advanced from the urethra into the bladder. When necessary, a smaller ureteroscope is further advanced from the bladder, through the ureter, and into the kidney. Alternatively, a nephroscope entering through a percutaneous tract into the kidney may be used in the kidney and upper urinary tract. In each of these protocols, endoscopes carry either an optical element or optical fiber bundle which in some cases is steerable so that individual stones or targeted tissue may be observed, captured, and removed from the kidney and bladder. Similarly, other types of endoscopes such as gastroscopes or colonoscopes are utilized for similar purposes in non-urinary body tracts or lumens. The endoscopes carry a working channel for the introduction of tools to the distal end of the device. The working channel in a smaller endoscope such as a ureteroscope or angioscope, however, has have a very small diameter, due to the very small size of the device itself, typically limiting tool use to one small tool at a time. Optionally, the stones or tissue may be fragmented using laser or other energy, and the intact stone and/or fragments may be removed using a deployable basket advanced through a working channel of the endoscope, while large or multiple tissue samples too large to be withdrawn through the working channel are managed similarly using baskets or grasping devices. In both cases, the entire endoscope must be withdrawn, along with the samples or fragments to in order to retrieve those for further study. This can require multiple placements and removals of the endoscope, extending the procedure duration as well as potentially increasing endoscopic-related trauma to the patient.

Endoscopic stone treatment is particularly difficult to perform in the bladder and kidney where the stones or stone fragments may be mobile and are present in a large open volume and thus are often difficult to capture. While the stones may be captured using a basket or other tools under direct visualization, steering the scope and firmly capturing the stone is problematic, particularly if the stone is mobile and suspended within the open volume. Moreover, if the stone is fragmented with energy, capturing the many stone fragments which disperse throughout the volume and can be even more difficult and time consuming.

For these reasons, it would be desirable to provide improved and alternative apparatus and protocols for the ureteroscopic treatment and removal of stones from the urinary system, particularly the kidney and bladder. Such systems and protocols will preferably be compatible with many or all conventional endoscopes which are commercially available. Desirably, the apparatus and protocols will facilitate capturing of the stones within the open volumes of the kidney and bladder, will allow for energy-based fragmentation of the stones while they remain captured, and will contain most or all of the stone fragments resulting from the fragmentation. The apparatus and protocols would preferably reduce the need to use the working channel of the endoscope, making the working channel available for an energy source or use in new protocols.

Similarly, tumor or growth debulking in a relatively large open volume is inefficient when multiple fragments are removed or also inefficient when multiple biopsy samples are taken due to the desirability of not having to remove the endoscope for each individual sample recovery. Despite the tissue being typically initially anchored to a lumen wall, recovery of loose sample is time consuming and inefficient. Further, in the case of a growth or tumor fully filling a smaller lumen such as the ureter, the target tissue may itself distort or mis-shape the lumen. Preferably the apparatus and protocols will help to correct this distortion and improve the ability of the endoscope to visualize and excise the tissue while aiding the collection and removal.

At least some of these objectives will be met by the inventions described below.

### 2. Background of the Background Art

This is a continuation in part of copending application Ser. No. 14/622,144. The present application claims the benefit of Provisional Application No. 61/118,802, filed on Dec. 1, 2008, and Provisional Application No. 61/170,055, filed on Apr. 16, 2009, which are incorporated in U.S. Pat. No. 8,986,291. Commonly owned, copending application Ser. Nos. 10/886,886; 11/777,515; 12/041,241; and 12/269,739 describe conformable structures which are deployable in the urethra and ureter and which may be used to entrap stones during lithotripsy. Dr. Bogdan Petrut has filed a Romanian Patent Application describing a stone capture device for attaching to a ureteroscope to capture and draw stones into a sheath for containing the stones while delivering laser energy to break up the stones. See also U.S. Pat. Nos. 3,760,810; 3,870,048; 4,222,308; 4,257,420; 4,471,766; 4,735,194; 5,423,834; 5,507,797; 6,099,535; 6,645,195; 6,869,395; 7,204,804; and 7,223,230. U.S. Patent Publication No. US2006/0116693 describes a stone capture device intended for use in lithotripsy treatment. The RothNet® foreign body retrieval device is described at http://www.usendoscopy.com/foreignbody.php.

### SUMMARY OF THE INVENTION

In a first aspect of the present invention, methods are provided for removing urinary stones or tissue from a body cavity, such as a kidney or, bladder or stomach, or a stone or tissue from a body lumen such as ureter, hepatic duct or colon. The methods comprise introducing a viewing scope, such as a commercially available ureteroscope, having an optical element at its end into an open volume of the body cavity. A perforate sweeping structure is deployed from a distal end of the viewing scope, while the viewing scope is steered and advanced within the open volume of the body cavity to engage the deployed perforate sweeping structure against the stone. Once the stone is engaged, the sweeping structure is further advanced to urge the stone against a wall structure of the body cavity so that the stone is captured between the sweeping structure and the wall structure. Energy is then applied through the viewing scope, typically laser energy delivered via an optical (laser) fiber advanced through a working channel of the viewing scope. The delivered energy disrupts the captured stone and large fragments, producing smaller stone fragments. Usually, the stone is held in place while delivering energy solely by the sweeping structure against the wall structure, and no separate basket, forceps, loop structures, or the like, are used to hold the stone in place. In a preferred aspect of the method, the region around the captured stone is irrigated, typically before, during, and/or after the energy-based fragmentation, to wash the stone fragments into the sweeping structure. Preferred sweeping structures comprise mesh structures, often double-walled mesh structures, in which the stone fragments become entrapped as they are washed away by the irrigating solution.

The viewing scope is usually introduced transluminally, i.e., through the urethra and optionally through the bladder, ureter, and into the kidney. Alternatively, however, the sweeping structure and viewing scope could be introduced through a percutaneous incision in the abdomen.

During delivery and prior to deployment, the perforate sweeping structure is usually maintained in a tubular configuration. Such a tubular structure may be deployed by axial foreshortening. The sweeping structure is disposed over a distal portion of the viewing scope, where the tubular configuration is transformed into a concave structure which extends distally from the distal end of the viewing scope. The concave structure, which may be conical, hemispherical, or have other expansibly tapered structures, will surround the optical element of the viewing scope so that view from the element is not obscured. Moreover, the tubular configuration of the sweeping structure will typically be sufficiently flexible so that the distal end or region of the viewing scope can be steered in a conventional manner without excessive constraint by the sweeping structure. When the tubular configuration of the sweeping structure is shifted to the concave structure, however, it will become more rigid, allowing it to engage, move, and entrap kidney stones against the body cavity wall while retaining sufficient flexibility to conform to an irregularly shaped wall surface. While a preferred perforate sweeping structure is deployed by foreshortening, alternative sweeping structures may have an initial collapsed, closed configuration extending over the distal end of the viewing structure and may be deployed or otherwise opened to a concave configuration surrounding the distal end of the viewing structure, typically by releasing the constrained structure from a surrounding sleeve or other structure.

The methods of the present invention optionally include steering the viewing scope within the body cavity while the distal portion of the viewing scope is present within the tubular configuration of the sweeping structure. Methods further comprise engaging the perforate structure against the urinary stones when said structure is sufficiently rigid to manipulate the stones while remaining sufficiently flexible to conform an outer rim of the structure to an irregularly shaped bladder wall and sufficiently porous to allow fluids to pass freely through the perforations or apertures in the perforate structure when the region is being irrigated. Generally, the perforate sweeping structure will be formed as a metal or polymeric mesh with individual interwoven wires or filaments. The mesh will have openings or interstices which are sufficiently large to allow the free flow of irrigation fluid, but which have dimensions which contain and/or entrap the stone fragments within the mesh, particularly within a double walled mesh structure which will be described hereinbelow. Usually, porosity of the deployed perforate structure will be sufficient to limit stones larger than 2 mm in any dimension from passing therethrough. The deployed perforate structure utilizes irrigation from the viewing scope to maintain the captured stone fragments (typically smaller than 2 mm) against the interior of the perforate structure and/or against the wall of the body cavity so as not to obscure vision during lithotripsy procedures.

Usually, the perforate sweeping structure will be removably attached to the distal end of the viewing scope. Thus, after the assembly viewing scope and perforate structure has been used in a procedure, the perforate sweeping structure may be detached from the assembly and disposed of while the viewing scope may be sterilized and reused.

While the presently preferred perforate sweeping structure will be an evertable tubular structure, as described above, other embodiments of the perforate sweeping structure include a self-expanding tube or other elongate structure which can be distally advanced from a carrier sleeve or sheath. Usually, the carrier sleeve or sheath will be configured as a “monorail” device which has a relatively short engagement length, typically from 2 cm to 10 cm, usually from 3 cm to 8 cm, with a lumen or passage therethrough which receives the viewing scope. Thus, the carrier sleeve may be advanced over a proximal end of the viewing scope in a manner similar to a monorail vascular catheter. A hypotube or other elongate shaft is attached at the proximal end of the carrier sleeve allowing the carrier sleeve to be pushed over the viewing scope until the sleeve reaches the distal end of the scope. At that point, a second pusher rod or element is used to distally advance a conical (tapered to open in the distal direction) or other expanding mesh structure from the carrier sleeve, where the structure will be configured so that a proximal end is aligned with the optical viewing element of the viewing scope. The assembly of the viewing scope and the carrier sleeve with deployed perforate sweeping structure then can be manipulated and advanced within the open volume of the body cavity to capture stones and engage the stones against a structure, typically a wall of the body lumen, prior to delivering energy to break up the stones. After the stones are broken up, the perforate structure may be proximally retracted so that the structure as well as the stones carried therein are drawn back into the carrier sleeve which may then be withdrawn from the body cavity to remove the stone fragments.

In a second aspect of the present invention, a stone capture device for use with a steerable viewing scope comprises a sheath and a perforate sweeping structure. The sheath has a distal end, a proximal end, and a lumen therebetween, where the lumen is positionable over the distal end of the viewing scope. Usually, the sheath will have a length which extends over most, but not all of the length of the viewing structure, allowing a proximal end of the sheath to be available for manipulation by the treating physician during a procedure. That is, the sheath will have a length sufficient to allow the proximal end of the sheath to lie outside of the patient even when the distal end has been fully advanced into the kidney. Usually, the sheath length will be in the range from 20 cm to 100 cm, more usually from 60 cm to 70 cm when configured for use with a relatively long ureteroscope, and will be in the range from 10 cm to 50 cm, usually from 30 cm to 35 cm when configured to work with a shorter cystoscope or nephroscope.

The perforate sweeping structure will be removably attached to and extend distally from the distal end of the sheath. The perforate sweeping structure will be shiftable between a tubular configuration with a width generally about the same as that of the sheath and a concave configuration which increases in width in the distal direction. A distal end of the viewing scope is disposed within the concave sweeping structure when said sweeping structure is deployed. Prior to deployment, the sweeping structure may be positioned proximally to the distal end of the viewing scope to enhance visibility and/or maneuverability of the distal scope tip. Usually, an optical element of the viewing scope will be generally centered within the deployed sweeping structure. Optionally, the stone capture device may further comprise a means for closing a distal end of the deployed concave sweeping structure to capture the stones therein. Conveniently, the closing means may be a simple wire, suture, or other loop or tether which extends around the distal end of the deployed concave sweeping structure. Thus, the tether can be drawn to close the distal end in the manner of a “purse string.” The sweeping structure typically comprises woven or braided filaments which form a mesh tube which can be foreshortened to evert to form a double-walled concave structure. The double-walled concave structure will usually have a conical or hemispherical geometry, typically being advanceable over the viewing scope, typically in a monorail fashion, the position the sweeping structure in the bladder or kidney.

In a preferred configuration where the perforate sweeping structure is attached to the distal end of the viewing scope, the sheath will typically be configured to position the perforate sweeping structure distally over a distal region of the viewing scope while the sweeping structure is in its tubular configuration. By drawing the viewing scope proximally, where the viewing scope is connected to a distal end of the tubular sweeping structure, the tubular configuration of the sweeping structure will be deformed to assume a concave configuration where all or a principal portion of the concave configuration is positioned distally of the distal end of the viewing structure. In its tubular configuration, the sweeping structure will be sufficiently flexible and bendable to allow steering of the viewing structure within the sweeping structure prior to deployment. After deployment into its concave configuration, however, the sweeping structure will assume its double-walled configuration and will have sufficient stiffness to engage and manipulate stones within the body cavity so that the stones may be urged against a cavity wall. Typically, the sweeping structure in its concave configuration will have a bending stiffness which is at least 25% greater than that in the tubular configuration, usually being at least 50% greater, and often being 100% greater or more. The sweeping structure in its concave configuration will further have a width which is greater than that of the sheath, typically being at least twice that of the sheath, often up to eight-fold larger that the sheath width.

The stone capture device is typically packaged as a kit where the sheath and perforate sweeping structure are attached to each other, sterilized, and present in a sterile package, such as a bag, tube, or box. The stone capture device is thus ready to be removably attached to the distal end of a conventional or commercially available viewing scope, such as a ureteroscope, where a distal portion of the scope is placed within the perforate sweeping structure of the capture device. The distal end of the scope will typically be secured to the distal tip of the tubular configuration of the perforate sweeping structure so that retraction of the viewing scope relative to the sheath will foreshorten the perforate sweeping structure causing it to assume the concave configuration.

Similarly, the same device structure may be utilized in other open lumens of the body such as the stomach or peritoneum for management of tissue removed from the body for the purposes of debulking a cancerous or non-cancerous growth or for collection of multiple or large biopsy samples.

In a final aspect, the same device structure may be utilized to expand and to circularize a body duct distorted by a stone or a growth in order to achieve better visualization and access for a debulking or biopsy procedure. In this case, the user would manipulate the endoscope until it was immediately adjacent to the restriction, then evert or expand the porous collection cone of the above device in order to correct the distortion caused by the restriction itself. In some cases, the deployment of the cone will simultaneously slightly expand the duct immediately adjacent to the restriction. Increased visualization, due to the expanded space, as well as the increased irrigation flow allowed through the porous cone improves visualization, particularly if the excision or biopsy process causes bleeding. A relatively large quantity of tissue may be removed in a single endoscopic placement by filling the cone before endoscope removal.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a stone capture device constructed in accordance with the principles of the present invention.

FIG. 2 is a perspective view of a viewing scope in the form of a commercially available ureteroscope having a steerable distal end.

FIG. 3 is a detailed view of the distal end of the stone capture device of the present invention.

FIGS. 4A and 4B illustrate the distal end of the stone capture device with a partially deployed sweeping structure.

FIGS. 5A and 5B illustrate the distal end of the stone capture device with a fully deployed sweeping structure.

FIG. 6 illustrates the initial placement of the stone capture device and viewing scope of the present invention through the urinary tract into a kidney.

FIGS. 7A-7D illustrate deployment of the sweeping structure on the stone capture device for capturing, fragmenting, and removing a stone within the kidney.

FIGS. 8A-8C illustrate the structure and use of an alternative embodiment of the sweeping structure of the present invention.

FIG. 9 illustrates the use of the stone capture device in a bladder.

FIGS. 10A-10B illustrates the use of the stone capture device in a duct or vessel.

### DETAILED DESCRIPTION OF THE INVENTION

Referring to FIG. 1, a stone capture device 10 constructed in accordance with the principles of the present invention comprises a sheath 12 having a proximal end 14 and a distal end 16. A deployable sweeping structure 18 is disposed at the distal end of the sheath 12 and is shiftable between a low profile tubular configuration (shown in full line) and an open or deployed concave configuration (shown in broken line). A hub 20 is typically provided at the proximal end 14 of the sheath 12.

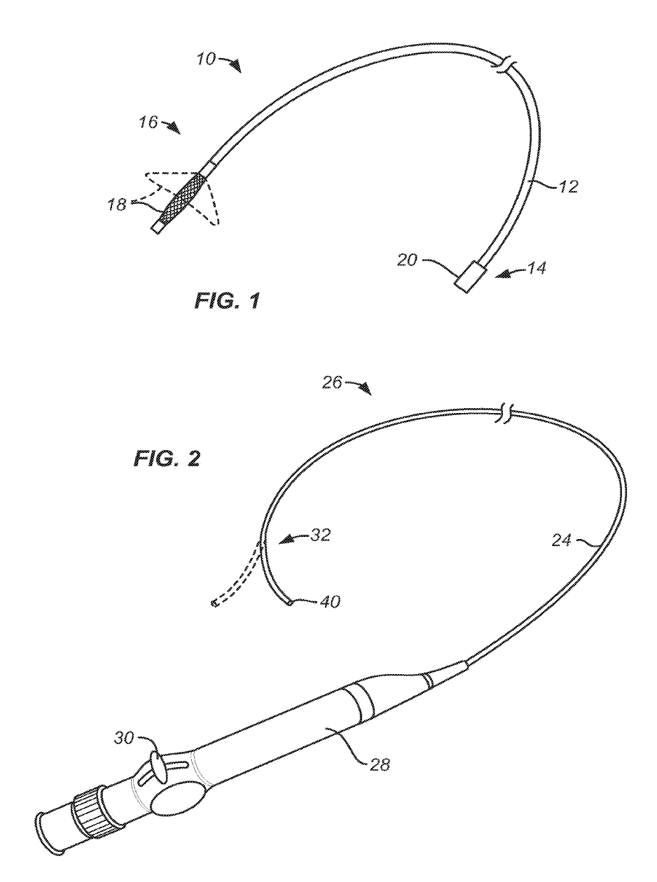

The stone capture device 10 of the present invention is intended to be mounted over the steerable shaft 24 of a conventional viewing scope (26, FIG. 2), typically a ureteroscope, such as those commercially available from Olympus/ACMI. The viewing scope 26 further comprises a control handle 28 attached to a proximal end of the shaft 24. The control handle 28 includes a steering lever 30 which can be advanced and retracted to selectively deflect or “steer” a distal region 32 of the shaft 24, as shown in broken line. The control handle 28 will further include a connector for compiling a light source and an optical element of the viewing scope to a suitable screen and power source (not shown).

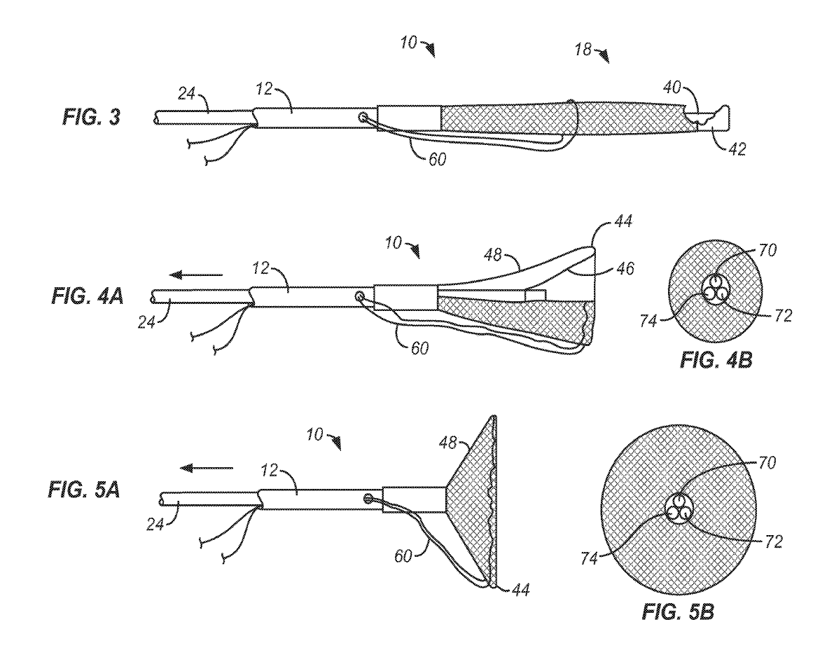

As shown in FIG. 3, a distal tip 40 of the steerable shaft 24 of the viewing scope 26 is introduced through the entire length of the sheath 12 of the stone capture device 10 so that said distal end is received in and attached to a distal tip 42 of the deployable sweeping structure. The deployable sweeping structure 18 is typically a woven or braided mesh, typically formed from polymeric or wire filaments or fibers, so that axial compression of the sweeping structure 18 from its tubular configuration (illustrated in FIG. 3) to a foreshortened configuration (as shown in FIGS. 4A and 5A, will cause the sweeping structure to assume a concave configuration, more particularly a conical configuration. Usually, the sweeping structure will be fabricated so that it preferentially bends and everts about a weakened region 44, as shown in FIGS. 4A and 5A. Thus, a distal most portion 46 of the sweeping structure 18 will preferentially evert into a proximal most portion 48. Thus, as the steerable shaft 24 is progressively pulled proximally, as shown by the arrows in FIGS. 4A and 5A, the steering structure progressively shortens with the forward diameter or width becoming larger.

The sweeping structure in its tubular configuration, as shown in FIG. 3, has only a single wall and is thus relatively flexible, typically having a bending stiffness in the ranges set forth above. As the sweeping structure 18 is everted, however, the wall becomes doubled, increasing the bending stiffness within the ranges set forth above. The relatively flexible tubular configuration is desirable since it will be over the steerable distal region 32 of the viewing scope. Thus, the viewing scope may be steered even while the sweeping structure 18 is over it. When the sweeping structure is deployed, however, it will have more stiffness and rigidity to facilitate engaging and manipulating the urinary stones within the kidney or bladder. As the deployed sweeping structure is distal to the distal end of the steerable shaft 24, however, the structure will not interfere with steering and advancement.

The sweeping structure mesh is preferably constructed from a wire braid. Usually eight to 36 wires are used to construct braid. The wire will usually be formed from a flexible metal or polymer material such as a superelastic nickel-titanium alloy (nitinol), a nylon, or a polyethylene terephthalate (PET).

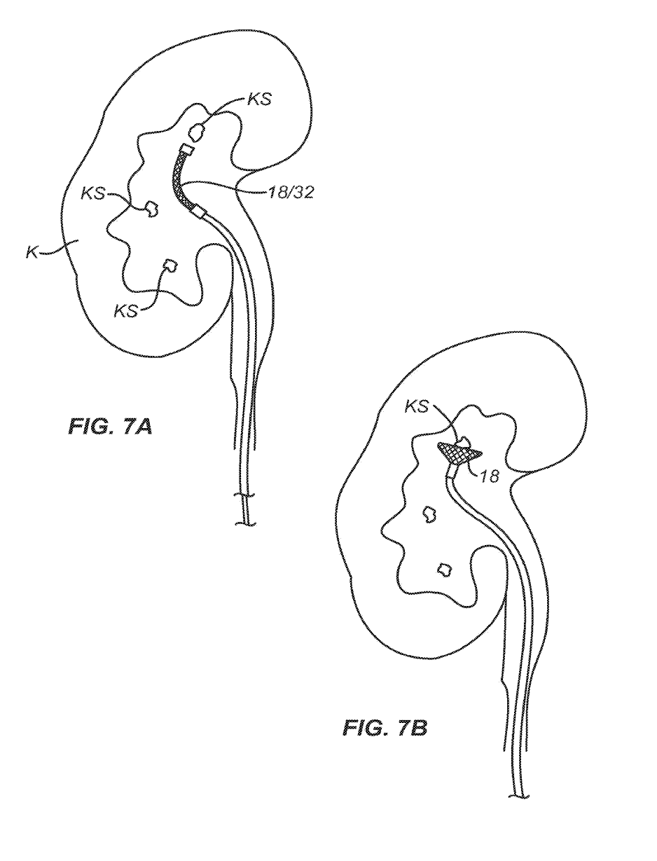

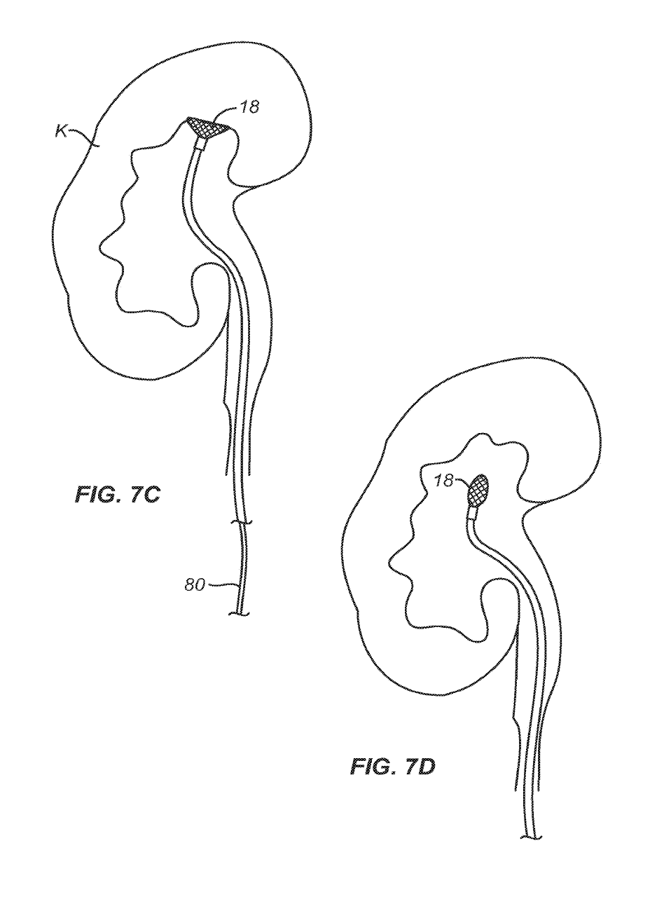

The sweeping structure 18 will further include a mechanism for drawing the open end closed after it has been partially or fully deployed. In the exemplary embodiments of FIGS. 3, 4A-B, and 5A-B, the structure to close the end is a simple filament or suture 60 which passes through the mid-section of the sweeping structure which becomes the forward edge of the sweeping structure after deployment. Thus, by pulling on the filament, the filament acts as a purse string to draw the sweeping structure closed, as illustrated in FIG. 7D discussed below.

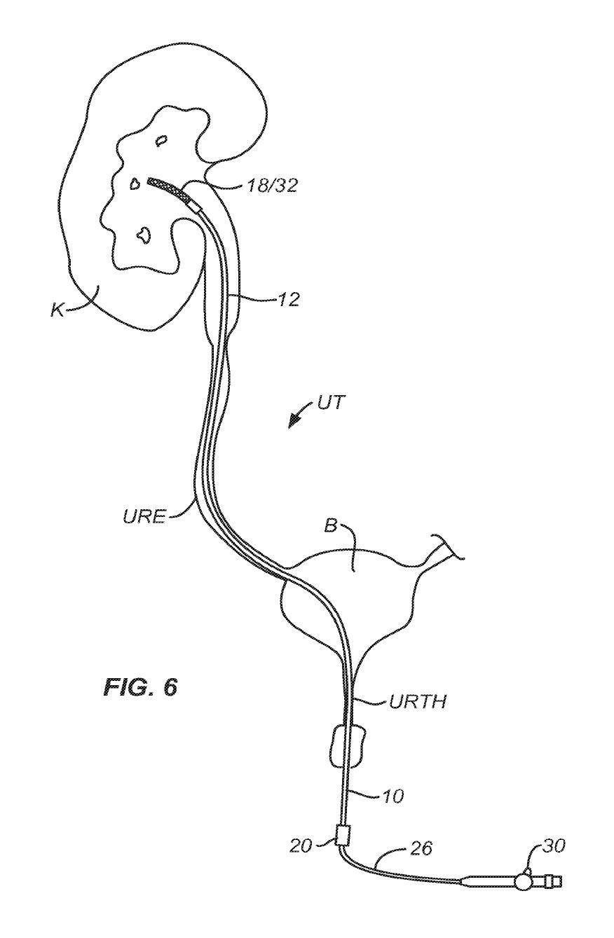

The viewing scope 26 will be introduced into the urinary tract UT with a stone capture device 10 disposed thereover, as illustrated in FIG. 6. The assembly of the viewing scope 26 and the stone capture device 10 is introduced in the same manner as a conventional ureteroscope without the stone capture device. That is, a distal end of the assembly of the present invention will be introduced through the urethra URTH into the bladder B, and optionally further through the ureter URE and into the kidney K. Once in the kidney, the non-deployed sweeping structure 18 and the distal region 32 of the viewing scope may be steered together using the lever 30 of the viewing scope which selectively deflects a distal portion of the shaft 24 of the viewing scope 26.

As illustrated in FIG. 7A, the kidney K may have a number of kidney stones KS therein. The sweeping structure 18 and distal region 32 may be steered while the physician views through the optical element 70 of the viewing scope (illustrated in FIGS. 4B and 5B). While viewing, illumination will be provided by a light source 72 on the viewing scope. Once a target kidney stone KS is located, as shown in FIG. 7A, the sweeping structure 18 may be deployed, as shown in FIG. 7B. The combination stone capture device 10 and viewing scope 26 (FIG. 2) may then be advanced, while continuing to optically view the region, until the deployed sweeping structure 18 engages a wall of the kidney, as shown in FIG. 7C, to capture the kidney stone between the sweeping structure and the wall. An optical (laser) fiber 80 or other stone fragmentation device may then be introduced through the working channel 74 (FIGS. 4B and 5B) of the viewing scope and advanced into the region where the stone is captured. By applying laser or other energy to the stone, the stone will be fragmented. Preferably, irrigation medium will be introduced simultaneously through the working channel to sweep the resulting stone fragments into the double-walled mesh of the deployed sweeping structure, thus simultaneously clearing the scope's field of view and entrapping the stone fragments therein. Occasionally, stones in the kidney are trapped within one of the cul-de-sac-like calices of the kidney. The sweeping structure may be advantageously deployed over the mouth of such a calyx to contain the stone and fragments produced by the lithotripsy process, in exactly the same manner as above. After the stone has been fragmented sufficiently, and the stone fragments captured, the filaments 60 may be retracted in order to close the sweeping structure 18 over the stone fragments, as shown in FIG. 7D. At that point, the combined assembly of the stone capture device 10 and the viewing scope 26 may be withdrawn from the urinary tract. After the procedure is complete, the stone capture device 10 may be removed from the viewing scope 26, and the stone capture device will usually be disposed of in a conventional manner. The viewing scope, however, can be sterilized and reused in subsequent procedures.

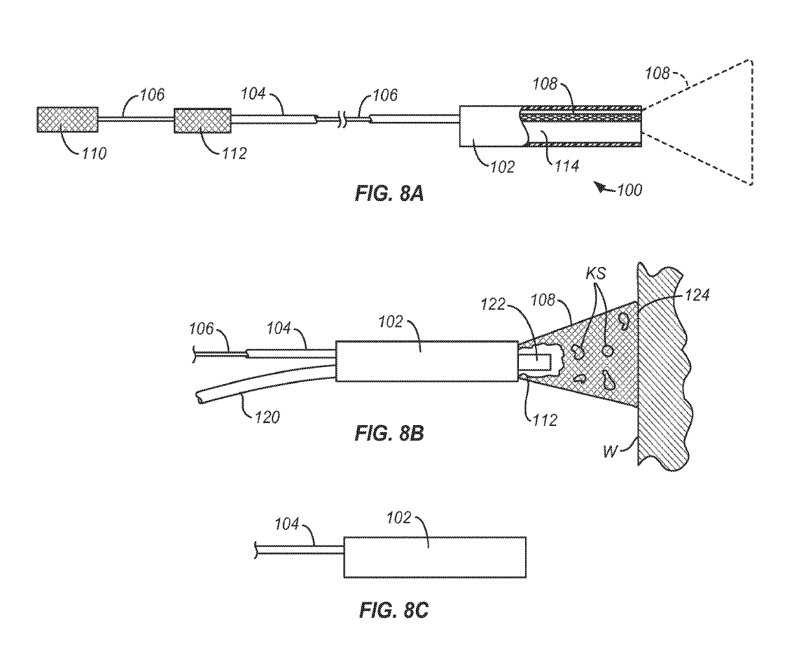

Referring now to FIGS. 8A-8C, an alternative perforate sweeping structure 100 comprises a carrier sleeve 102 attached at the distal end of a hypotube or other elongate tubular member 104. A pusher rod 106 extends coaxially through an inner passage or lumen of the hypotube 104 and is attached to a perforate sweeping structure 108 which is in a proximally retracted, radially collapsed configuration as shown in FIG. 8A. By pushing on the rod 106, typically using handle 110 while holding on to a second handle 112 which is attached at the proximal end of the hypotube 104, the self-expanding perforate sweeping structure 108 can be distally advanced so that it is released from the sleeve 102 and expands into an open conical configuration, as shown in FIG. 8B. A proximal end 112 of the perforate sweeping structure 108 will also expand to open and align with a passage or lumen 114 (FIG. 8A) which runs through the carrier sleeve 102.

The sweeping structure assembly 100 may then advance over a viewing scope 120 (FIG. 8B) by advancing a proximal end of the scope into the passage 114 of the sleeve 102. The hypotube 104 may be used to distally advance the sleeve 102 over the viewing structure 120 until the sleeve 102 approaches a distal end 122 of the viewing scope 120, as best seen in FIG. 8B. At that point, the viewing scope 120 and/or the assembly of sleeve 102 and hypotube 104 may be manipulated to cause the deployed perforate sweeping structure 108 to capture kidney stones KS therein. By then engaging a distal periphery 124 of the perforate sweeping structure 108 against the wall W, of the body cavity, kidney stones KS may be captured and energy applied through an optical fiber advanced through a working channel of the viewing scope 120 in order to disrupt the stones. After the stones are disrupted into a plurality of small fragments, the hypotube 104 may be retracted proximally in order to draw and collapse the sweeping structure 108 back into the passage 114 of sleeve 102 (as shown in FIG. 8C), optionally after the viewing scope 120 has been at least partially retracted therein. The viewing scope and sweeping structure assembly 100 may then be withdrawn from the body cavity to remove the stones.

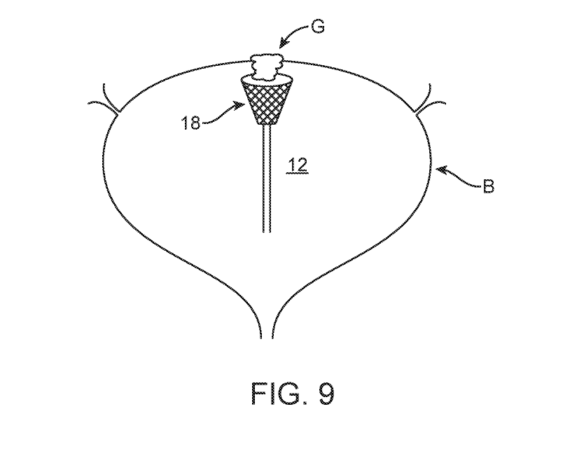

FIG. 9 illustrates the use of the stone capture device 10 in the bladder B for retention and removal of a bladder stone BS or a growth G or malignancy. The device 10 is utilized in a similar manner as in the kidney K as illustrated in FIG. 7A-D. The everted porous cone 18 is not utilized as a sweeping structure, but is still utilized as a containment device to isolate and to trap tissue fragments removed during debulking. In the case of biopsy rather than removal or debulking, the containment structure 18 is similarly used to isolate the region to be sampled, then will serve to collect those samples for simultaneous removal.

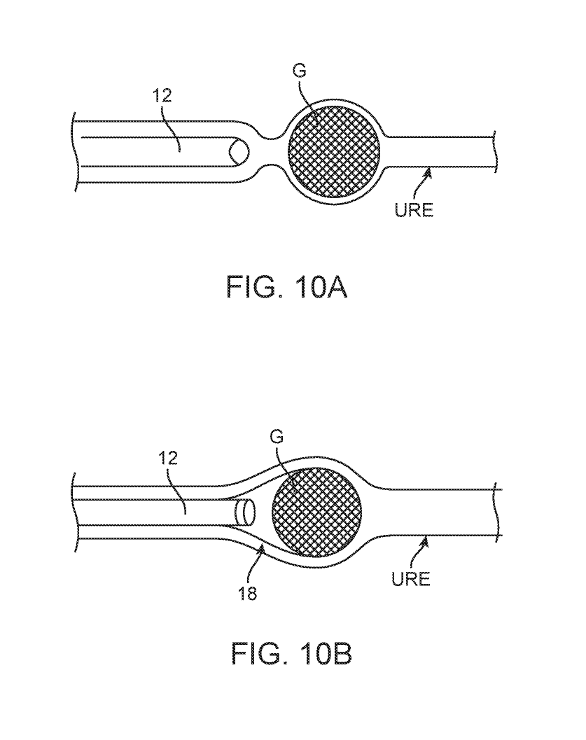

FIG. 10 illustrates the use of the device 10 in a duct or vessel such as a ureter, colon, hepatic duct, airway, or blood vessel where there is not a large body cavity relative to the endoscope. In this case, the device 10 is shown in use in the ureter URE where there is situated a growth G or stone S shown completely obstructing the duct lumen. FIG. 10A illustrates the growth G distorting the ureter URE, so that the ureter is partly obstructed adjacent to the growth, not allowing good access by ureteroscope 12 to growth G. FIG. 10B illustrates the deployment of everted porous cone 18, again as a containment structure but also as a dilation structure to improve visualization and access to growth G. Once the procedure is completed, multiple fragments may be removed simultaneously as in FIG. 7D.

While the above is a complete description of the preferred embodiments of the invention, various alternatives, modifications, and equivalents may be used. Therefore, the above description should not be taken as limiting the scope of the invention which is defined by the appended claims.

1. A method for controlling tissue during endoscopic debulking or biopsy of a body lumen, said method comprising:
mounting a perforate containment structure over a distal end of a viewing scope introduced into the body lumen;
deploying the perforate containment structure distally from the viewing scope so as to form a conical shape forward of the viewing element and working channel of the scope, the deployed perforate containment structure having an open distal end;
positioning the scope adjacent a growth or target tissue; and
fragmenting or
excising the growth or target tissue through or with the scope, while containing at least some of the fragmented or excised growth or target tissue within the containment structure.

2. A method as in claim 1, wherein irrigation is employed to clear vision and to position at least a portion the growth or target tissue against the perforate containment structure.

3. A method as in claim 1, wherein fragmenting or excising the target tissue comprises taking multiple biopsies.

4. A method as in claim 1, wherein a diameter of the deployed perforate containment member is larger than an outside diameter of the scope.

5. A method as in claim 4, wherein the diameter of the deployed perforate containment member is at least twice the outside diameter of the scope.

6. A method as in claim 1, wherein the body lumen is the stomach, kidney, bladder, or peritoneal cavity.

7. A method as in claim 1, further comprising closing the open distal end of the deployed perforate containment structure with the fragmented or excised growth or target tissue contained therein, wherein closing the distal end of the containment structure comprises reducing a diameter of the distal end.

8. A method as in claim 7, wherein the open distal end of the deployed perforate containment structure is closed prior to the fragmented or excised growth or target tissue being removed through or with the scope.

9. A method as in claim 7, wherein closing the open distal end of the deployed perforate containment structure comprises drawing a tether coupled to the open distal end.

10. A method as in claim 9, wherein the tether passes through a mid-section of the perforate containment structure before coupling to the distal end of the perforate containment structure.

11. A method as in claim 1, wherein deploying the perforate containment structure comprises dilating the body lumen.

12. A method as in claim 1, wherein positioning the scope adjacent the growth or target tissue comprises passing the growth or target tissue through the open distal end of the deployed perforate containment structure to be contained within the cone of the perforate containment structure.

13. A method for controlling tissue during endoscopic debulking or biopsy of a body duct or vessel, said method comprising:
mounting a perforate containment structure over a distal end of a viewing scope introduced into the body duct or vessel lumen with a partial or full occlusion;
deploying the perforate containment structure distally from the viewing scope so as to form a conical shape forward of the viewing element and working channel of the scope, the conical shape being at least the full diameter of the lumen, the deployed perforate containment structure in the conical shape having an open distal end, and the deployed perforate containment structure expanding or dilating a region of the body duct or vessel between the tip of the scope and the target growth or tissue; and
fragmenting or
excising the growth or target tissue through the scope, while containing at least some of the fragmented or excised growth or target tissue within the containment structure, the fragmented or excised growth or target tissue having been passed into the deployed perforate containment structure through the open distal end before removal from the body duct or vessel.

14. A method as in claim 13, wherein the body duct or vessel is a ureter, colon, hepatic duct, airway, or blood vessel.

15. A method as in claim 13, further comprising closing the open distal end of the deployed perforate containment structure with the fragmented or excised growth or target tissue contained therein.

16. A method as in claim 15, wherein the open distal end of the deployed perforate containment structure is closed prior to the fragmented or excised growth or target tissue being removed from the body duct or vessel through or with the scope.

17. A method as in claim 16, wherein closing the open distal end of the deployed perforate containment structure comprises drawing a tether of the open distal end.

18. A method as in claim 17, wherein the tether passes through a mid-section of the perforate containment structure before coupling to the distal end of the perforate containment structure.

19. A method as in claim 13, wherein irrigation is employed to clear vision and to position the fragmented or excised growth or target tissue against the perforate containment structure.

20. A method as in claim 13, wherein fragmenting or excising the growth or target tissue comprises taking multiple biopsies.

21. A method as in claim 13, wherein a diameter of the deployed perforate containment member is larger than an outside diameter of the scope.

22. A method as in claim 21, wherein the diameter of the deployed perforate containment member is at least twice the outside diameter of the scope.

23. A method as in claim 13, further comprising removing the fragmented or excised growth or target tissue from the body duct or vessel through or with the viewing scope.

24. A method as in claim 23, wherein removing the fragmented or excised growth or target tissue comprises removing a first portion of the growth or target tissue while a second portion of the growth or target tissue remains contained within the containment structure.

25. A method as in claim 1, further comprising removing the fragmented or excised growth or target tissue from the body duct or vessel through or with the viewing scope.

26. A method as in claim 25, wherein removing the fragmented or excised growth or target tissue comprises removing a first portion of the growth or target tissue while a second portion of the growth or target tissue remains contained within the containment structure.
